(19) United States
(12) Patent Application Publication
(10) Pub. No.: US 2022/0386925 A1
(43) Pub. Date: Dec. 8, 2022

(54) SIGNAL PROCESSING CIRCUIT

(71) Applicant: SONY SEMICONDUCTOR SOLUTIONS CORPORATION, KANAGAWA (JP)

(72) Inventor: AKITO SEKIYA, TOKYO (JP)

(21) Appl. No.: 17/755,353

(22) PCT Filed: Sep. 25, 2020
(86) PCT No.: PCT/JP2020/036340

§ 371 Date: Apr. 27, 2022

(30) Foreign Application Priority Data
Nov. 6, 2019 (JP) 2019-201518

(51) Int. Cl. A61B 5/30; A61B 5/00

(52) U.S. Cl. CPC A61B 5/30; A61B 5/7228; A61B 5/7203

(57) ABSTRACT

A signal processing circuit in one aspect of the present disclosure includes a first circuit, a second circuit, an electric wire, and a third circuit. The first circuit has at least a first input terminal that receives a first signal and a first output terminal that outputs a second signal at least based on the first signal. The second circuit has at least a second input terminal that receives the second signal and a second output terminal that outputs a frequency-modulated second signal. The electric wire is electrically connected with the second output terminal. The third circuit has at least a third input terminal that receives the frequency-modulated second signal and a third output terminal that outputs a second signal demodulated to a frequency at the time of input to the first circuit. The electric wire is further electrically connected with other than the second output terminal and the third input terminal.

### TECHNICAL FIELD

The present disclosure relates to a signal processing circuit.

### BACKGROUND ART

In biopotential sensing for measuring brain waves, heartbeats, and the like, a countermeasure against hum noise contained in a signal obtained from an observation target is an important technology for realizing highly accurate sensing. As an effective conventional technology for preventing hum noise, for example, there is driven right leg (DRL) technology.

The DRL technology is technology that attempts to cancel hum noise generated in an observation target by outputting a signal containing hum noise to the observation target. Specifically, a first electrode for receiving a signal from the observation target and a second electrode for outputting a signal to the observation target are attached to the observation target. Generally, a signal indicating intermediate potential (common potential) between the signal obtained by the first electrode and a reference signal is input to the second electrode. Since the signal input to the second electrode is generated on the basis of the signal obtained by the first electrode, it contains hum noise that is the same as hum noise contained in the signal obtained by the first electrode. That is, the hum noise contained in the signal obtained by the first electrode is returned to the observation target via the second electrode. Therefore, the hum noise contained in the signal obtained by the first electrode is suppressed.

### CITATION LIST

### Non-Patent Document

Non-Patent Document 1: BRUCE B. WINTER et al, “Reduction of Interference Due to Common Mode Voltage in Biopotential Amplifiers”, IEEE TRANSACTIONS ON BIOMEDICAL ENGINEERING, JANUARY 1983, VOL. BME-30, NO. 1, P58-62.

### SUMMARY OF THE INVENTION

### Problems to be Solved by the Invention

As described above, the DRL technology has a dedicated line for transmitting the hum noise obtained by the first electrode to the second electrode in order to return the hum noise to the observation target. Normally, the first electrode and the second electrode are mounted at some distance from each other to avoid unnecessary influence. Therefore, the dedicated line has a certain length. Therefore, when the DRL technology is used, there is a possibility that the dedicated line impairs convenience of biopotential sensing. For example, when a sensing device is attached to the observation target, the dedicated line becomes an obstacle, which causes a problem that a degree of freedom of attachment is reduced.

The present disclosure provides a signal processing circuit or the like that eliminates need for a dedicated line to transmit a signal returned to an observation target.

### Solutions to Problems

A signal processing circuit in one aspect of the present disclosure includes a first circuit, a second circuit, an electric wire, and a third circuit. The first circuit has at least a first input terminal that receives a first signal and a first output terminal that outputs a second signal at least based on the first signal. The second circuit has at least a second input terminal that receives the second signal and a second output terminal that outputs a frequency-modulated second signal. The electric wire is electrically connected with the second output terminal. The third circuit has at least a third input terminal that receives the frequency-modulated second signal and a third output terminal that outputs a second signal demodulated to a frequency at the time of input to the first circuit. Then, the electric wire is further electrically connected with other than the second output terminal and the third input terminal.

Furthermore, it is also possible that the first signal is a signal obtained from an observation target and that the second signal is a signal output to the observation target.

Furthermore, the signal processing circuit may further include: a first electrode that receives the first signal from the observation target when attached to the observation target; and a second electrode that outputs the second signal to the observation target when attached to the observation target.

Furthermore, the electric wire may be further electrically connected with the third input terminal and a power supply.

Furthermore, it may be configured that the first circuit further has a fourth input terminal that receives a third signal and that the electric wire is further electrically connected with the third input terminal and the fourth input terminal.

Furthermore, it may be configured that the electric wire is further electrically connected with the first input terminal and that the third input terminal is electrically connected with the third output terminal.

Furthermore, it is possible that the first circuit further has a fourth input terminal that receives a third signal and a fourth output terminal that outputs a fourth signal based on the first signal and the third signal and that hum noise contained in the fourth signal is reduced as compared with that before the third signal is output to the second electrode.

Furthermore, it is possible to adopt a configuration of a measuring device including the signal processing circuit.

### BRIEF DESCRIPTION OF DRAWINGS

FIG. 1 is a block diagram showing a configuration example of a signal processing circuit according to an embodiment of the present disclosure.

FIG. 2 is a block diagram showing a configuration example of the signal processing circuit using conventional DRL technology.

FIG. 3 is a block diagram showing a configuration example of the signal processing circuit in a case where a power supply line is used as a transmission path.

FIG. 4 is a block diagram showing a configuration example of the signal processing circuit in a case where a reference signal transmission line is used as the transmission path.

FIG. 5 is a block diagram showing a configuration example of the signal processing circuit in a case where an observation target is used as the transmission path.

### MODE FOR CARRYING OUT THE INVENTION

Hereinafter, an embodiment of the present disclosure will be described with reference to the drawings.

### First Embodiment

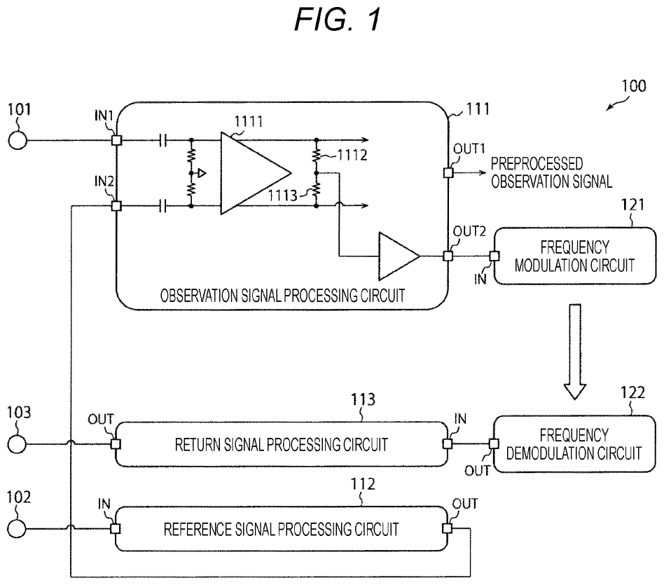

FIG. 1 is a block diagram showing a configuration example of a signal processing circuit according to an embodiment of the present disclosure. A signal processing circuit 100 according to the present embodiment includes an observation electrode (first electrode) 101, a reference electrode 102, a return electrode (second electrode) 103, an observation signal processing circuit (first circuit) 111, a reference signal processing circuit 112, a return signal processing circuit 113, a frequency modulation circuit (second circuit) 121, and a frequency demodulation circuit (third circuit) 122.

The signal processing circuit 100 is a circuit for obtaining a signal with suppressed hum noise from a predetermined observation target by returning hum noise contained in a signal obtained from the observation target to the observation target. The signal obtained from the observation target is described as an observation signal. Note that, in the present disclosure, the signal means an electric signal, and the observation signal indicates potential. The potential is described as observation potential. Furthermore, a signal returned to the observation target, in other words, a signal output from the signal processing circuit 100 to the observation target is described as a return signal (second signal).

Note that it is assumed that the observation target is a living body, but the observation target is not limited to the living body. Any object can be an observation target as long as the hum noise contained in the observation signal is suppressed by the signal processing circuit 100 of the present disclosure.

Note that, in each drawing of the present disclosure, an input terminal of each circuit is represented by IN, and an output terminal of each circuit is represented by OUT. That is, a signal input to each circuit is input via the input terminal of each circuit, and a signal output to each circuit is output via the output terminal of each circuit. Details will be described later.

Each component of the signal processing circuit 100 will be described.

The observation electrode 101 (first electrode) is attached to an observation target and detects observation potential. That is, the observation electrode 101 receives an observation signal (first signal) from the observation target when attached to the observation target.

The reference electrode 102 detects reference potential. That is, the reference electrode 102 receives a signal indicating the reference potential (third signal). The reference potential is reference potential for taking a difference from potential indicated by the observation signal. The signal indicating the reference potential is described as a reference signal.

In a case where a living body is the observation target, the reference electrode 102 is often attached to the living body, but the reference potential may be appropriately determined, and therefore, an attachment destination of the reference electrode 102 is not particularly limited. It may simply be attached to a device that outputs reference potential.

The return electrode 103 (second electrode) is attached to the observation target, and outputs a signal returned to the observation target from the return signal processing circuit 113 to the observation target. That is, the return electrode 103 outputs a return signal to the observation target when attached to the observation target.

The observation signal processing circuit 111 has at least two input terminals. One of the input terminals (IN1 of the observation signal processing circuit 111, a first input terminal) is connected with the observation electrode 101 and receives the observation signal from the observation electrode 101. Another input terminal (IN2 of the observation signal processing circuit 111, a fourth input terminal) is connected with the reference signal processing circuit 112 and receives the reference signal from the reference signal processing circuit 112.

Note that “connection” means electrical connection in the present disclosure. For example, connection of the input terminal of the observation signal processing circuit 111 with the observation electrode 101 means that a signal, that is, a current can be received from the observation electrode 101. Therefore, the description “connection” also includes connection via an electric wire or the like for transmitting a signal.

The observation signal processing circuit 111 performs preprocessing necessary for performing various processing using the observation signal. For example, the observation signal processing circuit 111 may adjust the observation potential by taking a difference from the reference potential. Furthermore, for example, since the observation potential is very small, an amplifier or the like may be included in the observation signal processing circuit 111 to amplify the observation potential. Note that, in the present embodiment, the use of the observation signal is not limited, and components in the observation signal processing circuit 111 may be different depending on the use.

The observation signal processing circuit 111 has at least two output terminals. One of the output terminals (OUT1 of the observation signal processing circuit 111, a fourth output terminal) outputs a signal (fourth signal) based on the observation signal and the reference signal. For example, it outputs a preprocessed observation signal adjusted by taking a difference between the observation potential and the reference potential. It is assumed that the output terminal is connected to a device or the like that performs various processing using the observation signal. Another output terminal (OUT2 of the observation signal processing circuit 111, a first output terminal) outputs a return signal.

It is sufficient if the return signal contains the same hum noise as the hum noise contained in the observation signal. For example, a signal showing intermediate potential (common potential) between the observation potential and the reference potential can be used as the return signal. In the example of FIG. 1, a configuration example in a case of receiving the common potential is shown in the observation signal processing circuit 111. In the example of FIG. 1, the observation signal processing circuit 111 includes an amplifier 1111. Furthermore, resistors 1112 and 1113 are connected to two output terminals of the amplifier, respectively, and these resistors are connected in series. Resistance values of these resistors are the same. In such a configuration, potential at a connection point of the resistors 1112 and 1113 is common potential. Therefore, by connecting a start end of the output terminal that outputs the return signal to the connection point, it is possible to output a signal indicating the common potential as the return signal from an end of the output terminal.

Note that an internal configuration of the observation signal processing circuit 111 is not limited to the example shown in FIG. 1. Furthermore, in the example of FIG. 1, one set of the observation electrode 101 and the observation signal processing circuit 111 is shown, but there may be a plurality of sets of the observation electrode 101 and the observation signal processing circuit 111. Furthermore, in a case where there is a plurality of the sets, only one of the plurality of observation signal processing circuits 111 may output a return signal.

The reference signal processing circuit 112 has at least one input terminal and at least one output terminal. The input terminal is connected with the reference electrode 102 and receives a reference signal from the reference electrode 102. The reference signal processing circuit 112 is a circuit for adjusting reference potential of the reference signal so that the observation signal processing circuit 111 can perform processing using the reference potential. Components in the reference signal processing circuit 112 may be appropriately changed depending on adjustment contents. In a case where it is not adjusted, only an electric wire that transmits the reference signal may be present in the reference signal processing circuit 112, or the electric wire may be provided with a diode for preventing backflow. The output terminal is connected with the input terminal of the observation signal processing circuit 111, and outputs the adjusted reference signal to the observation signal processing circuit 111.

The return signal processing circuit 113 has at least one input terminal and at least one output terminal. The input terminal receives the return signal from the observation signal processing circuit 111. Then, the return signal processing circuit 113 makes a necessary adjustment for outputting the return signal to the observation target. Components in the return signal processing circuit 113 may be appropriately changed according to adjustment contents. In a case where it is not adjusted, only an electric wire that transmits the return signal may be present in the return signal processing circuit 113, or the electric wire may be provided with a diode for preventing backflow. The output terminal is connected with the return electrode 103 and outputs the adjusted return signal to the return electrode 103.

The return signal contains the same hum noise as the hum noise contained in the observation electrode 101. Therefore, the hum noise is output to the observation target, and it is possible to cancel the hum noise contained in the signal acquired by the observation electrode 101. That is, the hum noise contained in the signal output from the observation signal processing circuit 111 to a device or the like that performs various processing using the observation signal is reduced as compared with that before the return signal is output to the return electrode 103.

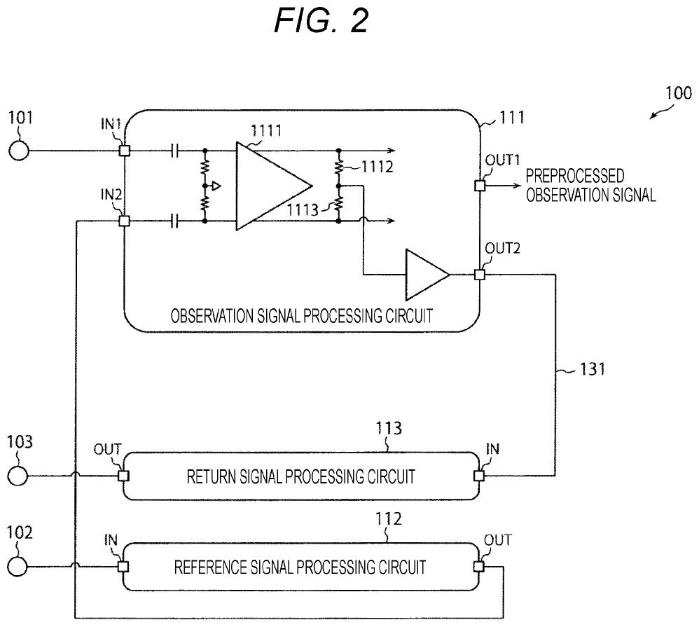

As described above, the return signal needs to be transmitted from the observation signal processing circuit 111 to the return signal processing circuit 113. FIG. 2 is a block diagram showing a configuration example of the signal processing circuit using conventional DRL technology. The conventional signal processing circuit has an electric wire dedicated to transmission of a return signal, which connects the observation signal processing circuit 111 and the return signal processing circuit 113. Hereinafter, the electric wire will be referred to as a return signal transmission line 131. The return signal is transmitted from the observation signal processing circuit 111 to the return signal processing circuit 113 through the return signal transmission line 131.

In the conventional signal processing circuit, length of the return signal transmission line 131 can be a problem. For example, the observation electrode 101 can be attached to a right wrist of a human body, the reference electrode 102 can be attached to a left wrist of the human body, and the return electrode 103 can be attached to a right ankle of the human body. Therefore, it seems that the length of the return signal transmission line 131 is short in FIG. 2, but in reality, the return signal transmission line 131 is long enough to impair convenience. For example, there arises a problem that a degree of freedom in mounting a sensing device is reduced.

Therefore, the signal processing circuit 100 of the present embodiment does not have the return signal transmission line 131, that is, the return signal dedicated line connected only to a terminal for outputting the return signal and a terminal for receiving the return signal. The return signal is transmitted from the observation signal processing circuit 111 to the return signal processing circuit 113 through a transmission path other than the return signal transmission line 131. Since the transmission path also transmits a signal other than the return signal, the signal processing circuit 100 includes a frequency modulation circuit 121 and a frequency demodulation circuit 122.

The frequency modulation circuit 121 has at least one input terminal and at least one output terminal. The input terminal is connected to the output terminal that outputs a return signal of the observation signal processing circuit 111, and receives the return signal. The frequency modulation circuit 121 modulates at least a frequency of the return signal. The transmission path other than the return signal transmission line 131 includes a signal other than the return signal. Therefore, frequency modulation is performed to separate the return signal from the other signal. The output terminal is connected to the transmission path and outputs a frequency-modulated return signal to the transmission path.

The frequency demodulation circuit 122 has at least one input terminal and at least one output terminal. The input terminal acquires a return signal from the transmission path other than the return signal transmission line 131. The frequency demodulation circuit 122 demodulates a frequency of the frequency-modulated return signal. That is, the frequency of the return signal is returned to a frequency at the time of input to the frequency modulation circuit 121. The output terminal is connected to the input terminal of the return signal processing circuit 113, and outputs a frequency-demodulated return signal to the return signal processing circuit 113.

The assumed transmission path will be described.

(First Transmission Path)

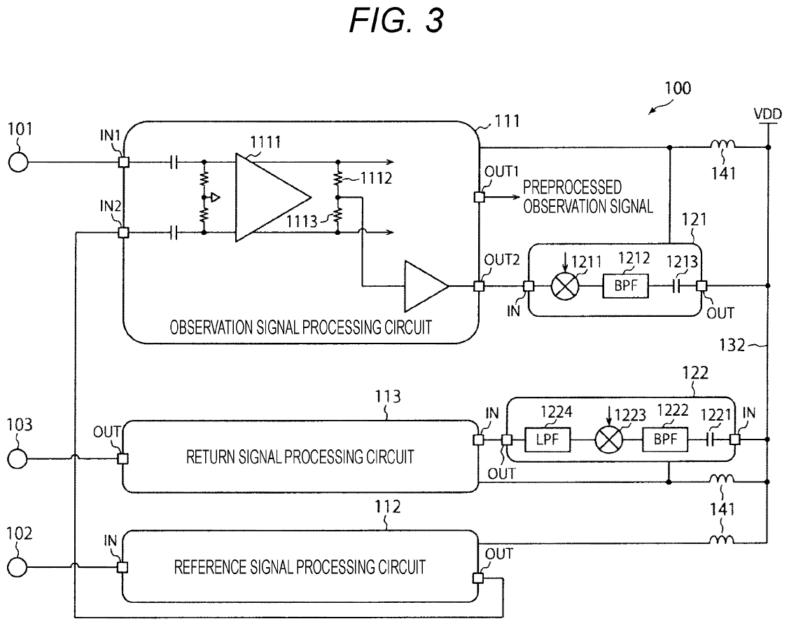

FIG. 3 is a block diagram showing a configuration example of the signal processing circuit in a case where a power supply line is used as the transmission path. That is, in the example of FIG. 3, a return signal is transmitted via a power supply line 132 instead of the return signal transmission line 131.

The power supply line 132 transmits drive power of each circuit in the signal processing circuit 100. Note that, in the example of FIG. 3, in order to distinguish from the transmission of the observation signal, the reference signal, and the return signal, IN sign is not assigned to connection with the power supply line related to supply of the drive power (that is, an input terminal that receives the drive power) to each circuit in the signal processing circuit 100.

Note that, as in the example of FIG. 3, power is not always output to each circuit by one power supply line 132. It is sufficient if the power supply line 132, to which the output terminal of the frequency modulation circuit 121 and the input terminal of the frequency demodulation circuit 122 are connected, is connected to one. For example, the reference signal processing circuit 112 may receive power from a power supply line other than the power supply line 132.

In the example of FIG. 3, it is assumed that current from a commercial power supply flows through the power supply line 132. That is, it is assumed that a direct current (DC) flows through the power supply line 132. On the other hand, the return signal corresponds to an alternating current (AC). Therefore, in the example of FIG. 3, the direct current from the power supply and the alternating current related to the return signal from the frequency modulation circuit 121 are superimposed. That is, the current flowing through the power supply line 132 can be said to be a superimposed signal including an AC component and a DC component.

An internal configuration of the frequency modulation circuit 121 and the frequency demodulation circuit 122 will be described. In the example of FIG. 3, the frequency modulation circuit 121 includes a mixer 1211, a bandpass filter (BPF) 1212, and a capacitor 1213. The mixer 1211 modulates (up-converts) the return signal input to the frequency modulation circuit 121 to a predetermined frequency higher than a present level on the basis of a reference frequency. A frequency band after the modulation, that is, the reference frequency for modulation is predetermined according to the assumed transmission path. Furthermore, the reference frequency may be received from the inside of the frequency modulation circuit 121 or may be received from the outside. The bandpass filter 1212 in the frequency modulation circuit 121 sets the modulated return signal as a signal of only a predetermined frequency band. The frequency band filtered by the bandpass filter 1212 may also be appropriately determined. The capacitor 1213 in the frequency modulation circuit 121 is connected to the power supply line 132, and superimposes the return signal on the current in the power supply line 132 by capacitive coupling.

In the example of FIG. 3, the frequency demodulation circuit 122 includes a capacitor 1221, a bandpass filter (BPF) 1222, a mixer 1223, and a low pass filter (LPF) 1224. The capacitor 1221 in the frequency demodulation circuit 122 is connected to the power supply line 132, and receives alternating current from the power supply line 132 by capacitive coupling. That is, the modulated return signal is received separately from the current of the power supply line 132. The bandpass filter 1222 removes unnecessary noise and the like from the modulated return signal, and makes the modulated return signal a signal of only a specific frequency band. The mixer 1223 demodulates (down-converts) the modulated return signal to a frequency at the time of input to the frequency modulation circuit 121 on the basis of a reference frequency for demodulation. A destination of the reference frequency for demodulation may be inside or outside the frequency modulation circuit 121. The roper filter 1224 of the frequency demodulation circuit 122 eliminates potential of the signal contained in the specific frequency band.

With such a configuration, the return signal is transmitted via the power supply line 132, and the conventionally required return signal transmission line 131 becomes unnecessary. Note that the internal configuration of the frequency modulation circuit 121 and the frequency demodulation circuit 122 is an example, and other components may be included. Furthermore, in the example of FIG. 3, a filter is provided in consideration of accuracy, but it is not essential and may be saved.

The return signal from the frequency demodulation circuit 122 also contains the same hum noise as the hum noise contained in the observation signal. Therefore, also in this configuration, it is possible to remove the hum noise similar to the case where the return signal is transmitted via the return signal transmission line 131 and input to the observation target.

Note that each circuit in the signal processing circuit 100 is connected with the power supply line 132 via an inductor in order to receive the direct current of the power supply line 132 as the power, that is, to cut the alternating current.

(Second Transmission Path)

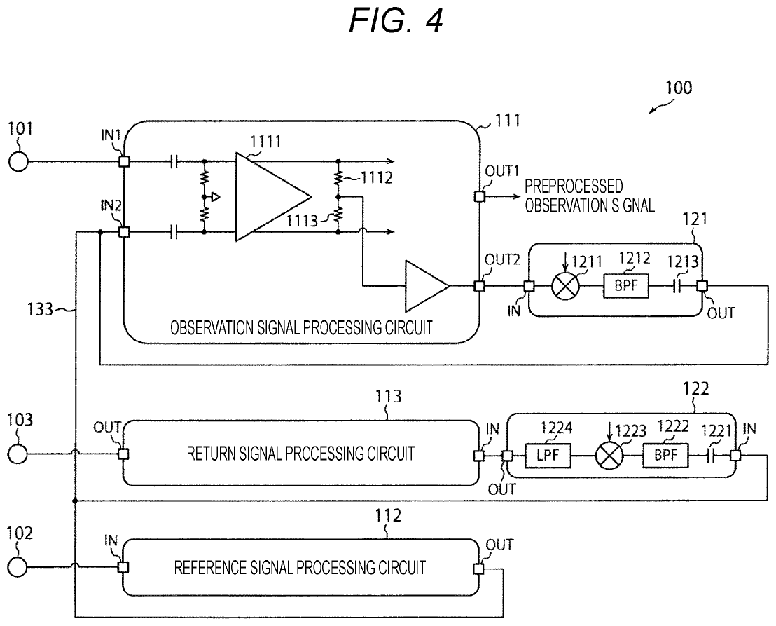

FIG. 4 is a block diagram showing a configuration example of the signal processing circuit in a case where a reference signal transmission line is used as the transmission path. In the example of FIG. 4, an electric wire for transmitting a reference signal is used as the transmission path. The electric wire is described as a reference signal transmission line 133. That is, a return signal is transmitted via the reference signal transmission line 133 instead of the return signal transmission line 131. Note that, in the example of FIG. 4, the power supply line 132 is omitted because it is not used for transmitting the return signal.

Since components in the frequency modulation circuit 121 and the frequency demodulation circuit 122 may be the same as those in the example of the first transmission path, description thereof will be omitted.

In the example of FIG. 4, the output terminal of the frequency modulation circuit 121 and the input terminal of the frequency demodulation circuit 122 are connected to the reference signal transmission line 133. Therefore, the return signal modulated from the output terminal of the frequency modulation circuit 121 is superimposed on a reference signal on the reference signal transmission line 133. A superimposed signal of the reference signal transmission line 133 is transmitted to the input terminal of the frequency demodulation circuit 122. Then, similarly to the example of the first transmission path, a demodulated return signal is output from the output terminal of the frequency demodulation circuit 122 and supplied to an observation target via the return signal processing circuit 113 and the return electrode 103.

With such a configuration, the return signal is transmitted via the reference signal transmission line 133, and the conventionally required return signal transmission line 131 becomes unnecessary.

(Third Transmission Path)

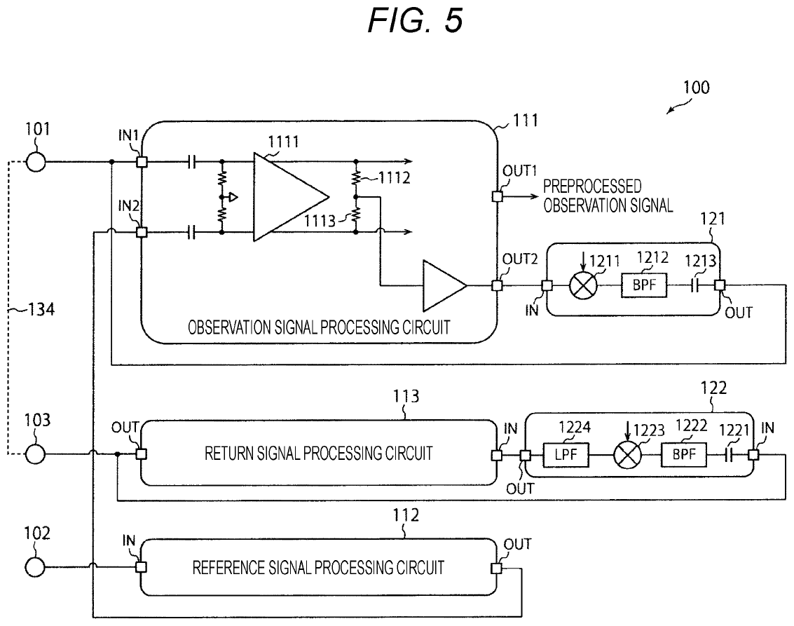

FIG. 5 is a block diagram showing a configuration example of the signal processing circuit in a case where an observation target is used as the transmission path. In the example of FIG. 5, the observation target to which the observation electrode 101 and the return electrode 103 are attached is regarded as the transmission line, and a return signal is transmitted via the observation target. Therefore, in the example of FIG. 5, the transmission path of the return signal does not exist in the signal processing circuit 100. Therefore, the transmission path of the return signal in the example of FIG. 5 is shown by a dotted line 134. Note that, in the example of FIG. 5, the power supply line 132 is omitted because it is not used for transmitting the return signal.

Since components in the frequency modulation circuit 121 and the frequency demodulation circuit 122 may be the same as those in the example of the first transmission path, description thereof will be omitted. The output terminal of the frequency modulation circuit 121 is connected with the observation electrode 101. Therefore, a modulated return signal is input to the observation target via the observation electrode 101. The input terminal of the frequency demodulation circuit 122 is connected with the return electrode 103. Therefore, the modulated return signal is input to the frequency demodulation circuit 122 from the observation target via the observation electrode 101. In other words, the return signal is extracted from the observation target by the return electrode 103 and input to the frequency demodulation circuit 122. The return signal input to the frequency demodulation circuit 122 is demodulated and input to the observation target via the return signal processing circuit 113 and the return electrode 103. That is, in a case where the observation target is used as the transmission path, the return electrode 103 plays two roles of extracting a frequency-modulated return signal and supplying a frequency-demodulated return signal.

As shown in the examples of FIGS. 3 to 5, the electric wire connected to the output terminal of the frequency modulation circuit 121 or the input terminal of the frequency demodulation circuit 122 is electrically connected with other than the output terminal and the input terminal. For example, the power supply line 132 and the reference signal transmission line 133 are connected with the observation signal processing circuit 111 and the like. Furthermore, in a case where the observation target is the transmission path, the electric wire, to which the output terminal of the frequency modulation circuit 121 is connected, is connected with the observation electrode 101 and the observation signal processing circuit 111, and the electric wire, to which the input terminal of the frequency demodulation circuit 122 is connected, is connected with the return electrode 103 and the return signal processing circuit 113. That is, in the examples of FIGS. 3 to 5, a dedicated line only for transmitting the return signal is not used.

As described above, according to the present embodiment, the return signal to be returned to the observation target can be transmitted to the return electrode 103 without using a dedicated line. This eliminates a need to separately provide a dedicated line for transmitting the return signal to the return electrode 103. That is, it is possible to eliminate wiring in question.

Note that, in the above, the observation signal processing circuit 111 and the frequency modulation circuit 121 are separated, but the frequency modulation circuit 121 may be incorporated in the observation signal processing circuit 111. Furthermore, although the return signal processing circuit 113 and the frequency demodulation circuit 122 are separated here, the frequency demodulation circuit 122 may be incorporated in the return signal processing circuit 113. The observation signal processing circuit 111 may be separated into a circuit for adjusting the observation signal and a circuit for receiving the return signal. As described above, each circuit shown in the present disclosure may include a plurality of finer circuits. Furthermore, there may be a circuit that collectively includes some of the circuits shown in the present disclosure.

The signal processing circuit 100 in the present embodiment can be used for various purposes. For example, it may be included in a measuring device for measuring potential of an observation target. For example, the measuring device may be configured so that the observation signal output from the observation signal processing circuit 111 is input to an AD (AC/DC) converter or the like and the observation signal converted by the AD converter is displayed via a monitor or the like.

Note that the above-described embodiment shows an example for embodying the present disclosure, and the present disclosure can be implemented in various other forms. For example, various modifications, substitutions, omissions, or combinations thereof are possible without departing from the gist of the present disclosure. A form in which such modifications, substitutions, omissions, and the like are made is also included in the scope of the present disclosure, and is similarly included in the invention described in the claims and an equivalent scope thereof.

Note that the present disclosure can have the following configurations.

[1]

A signal processing circuit including:

a first circuit having at least a first input terminal that receives a first signal and a first output terminal that outputs a second signal at least based on the first signal;

a second circuit having at least a second input terminal that receives the second signal and a second output terminal that outputs a frequency-modulated second signal;

an electric wire electrically connected with the second output terminal; and

a third circuit having at least a third input terminal that receives the frequency-modulated second signal and a third output terminal that outputs a second signal demodulated to a frequency at the time of input to the first circuit,

in which the electric wire is further electrically connected with other than the second output terminal and the third input terminal.

[2]

The signal processing circuit according to [1] described above,

in which the first signal is a signal obtained from an observation target, and

the second signal is a signal output to the observation target.

[3]

The signal processing circuit according to [1] or [2] described above, further including:

a first electrode that receives the first signal from the observation target when attached to the observation target; and

a second electrode that outputs the second signal to the observation target when attached to the observation target.

[4]

The signal processing circuit according to any one of [1] to [3] described above,

in which the electric wire is further electrically connected with the third input terminal and a power supply.

[5]

The signal processing circuit according to any one of [1] to [3] described above,

in which the first circuit further has a fourth input terminal that receives a third signal, and

the electric wire is further electrically connected with the third input terminal and the fourth input terminal.

[6]

The signal processing circuit according to [3] described above,

in which the electric wire is further electrically connected with the first input terminal, and

the third input terminal is electrically connected with the third output terminal.

[7]

The signal processing circuit according to [3] or [6] described above,

in which the first circuit further has a fourth input terminal that receives a third signal and a fourth output terminal that outputs a fourth signal based on the first signal and the third signal, and

hum noise contained in the fourth signal is reduced as compared with that before the third signal is output to the second electrode.

[8]

A measuring device including

the signal processing circuit according to any one of [1] to [7] described above.

### REFERENCE SIGNS LIST

100 Signal processing circuit
101 Observation electrode
102 Reference electrode
103 Return electrode
111 Observation signal processing circuit
1111 Amplifier
1112, 1113 Resistor
112 Reference signal processing circuit
113 Return signal processing circuit
121 Frequency modulation circuit
1211 Mixer
1212 Bandpass filter (BPF)
1213 Capacitor
122 Frequency demodulation circuit
1221 Capacitor
1222 Bandpass filter (BPF)
1223 Mixer
1224 Low Pass Filter (LPF)
131 Return signal transmission line
132 Power supply line
133 Reference signal transmission line
134 Observation target transmission path
141 Inductor
IN (IN1, IN2) Input terminal
OUT (OUT1, OUT2) Output terminal

1. A signal processing circuit comprising:
a first circuit having at least a first input terminal that receives a first signal and a first output terminal that outputs a second signal at least based on the first signal;
a second circuit having at least a second input terminal that receives the second signal and a second output terminal that outputs a frequency-modulated second signal;
an electric wire electrically connected with the second output terminal; and
a third circuit having at least a third input terminal that receives the frequency-modulated second signal and a third output terminal that outputs a second signal demodulated to a frequency at a time of input to the first circuit,
wherein the electric wire is further electrically connected with other than the second output terminal and the third input terminal.

2. The signal processing circuit according to claim 1,
wherein the first signal is a signal obtained from an observation target, and
the second signal is a signal output to the observation target.

3. The signal processing circuit according to claim 1, further comprising:
a first electrode that receives the first signal from the observation target when attached to the observation target; and
a second electrode that outputs the second signal to the observation target when attached to the observation target.

4. The signal processing circuit according to claim 1,
wherein the electric wire is further electrically connected with the third input terminal and a power supply.

5. The signal processing circuit according to claim 1,
wherein the first circuit further has a fourth input terminal that receives a third signal, and
the electric wire is further electrically connected with the third input terminal and the fourth input terminal.

6. The signal processing circuit according to claim 1,
wherein the electric wire is further electrically connected with the first input terminal, and
the third input terminal is electrically connected with the third output terminal.

7. The signal processing circuit according to claim 3,
wherein the first circuit further has a fourth input terminal that receives a third signal and a fourth output terminal that outputs a fourth signal based on the first signal and the third signal, and
hum noise contained in the fourth signal is reduced as compared with that before the third signal is output to the second electrode.
